(19) United States
(12) Patent Application Publication
(10) Pub. No.: US 2004/0049143 A1
(43) Pub. Date: Mar. 11, 2004

(54) Shoulder reduction device

(76) Inventor: David L. Short, Sedan, KS (US)

Correspondence Address: Kenneth W. Iles, Law Offices of Kenneth W. Iles, 9903 West 129th Street, Overland Park, KS, 66213-3222, US

(21) Appl. No.: 10/236,333

(22) Filed: Sep. 6, 2002

(51) Int. Cl. A61F 5/00

(52) U.S. Cl. 602/33

(57) ABSTRACT

A distal end of a staff is placed in the armpit under the dislocated shoulder while the proximal end is braced against the stomach of a skilled medical worker, who then pulls on a pulling end of a traction cord having a distal end connected to the patient's wrist by a wrist cuff. The traction cord may have a fixed end attached to the proximal end of the staff and may be operatively routed through a rachet mechanism, allowing the medical worker to relax during the procedure. A single person can reduce dislocated shoulders using the device.

### CROSS-REFERENCE TO RELATED APPLICATIONS

Not Applicable.

### STATEMENT REGARDING FEDERALLY SPONSORED RESEARCH OR DEVELOPMENT.

Not applicable.

### BACKGROUND OF THE INVENTION

1. Field of the Invention

The present invention is related to an apparatus for placing a shoulder joint in traction to reduce a dislocated shoulder joint or to stabilize a fractured humerus. More particularly, the present invention is related to a device that allows a single person to reduce the shoulder joint without assistance.

2. Description of the Related Art Including Information Disclosed Under 37 C.F.R. 1.97 and 1.98

The human shoulder joint consists of a ball in the upper arm bone, that is, humeral head, that is seated in a socket in the shoulder, that is the glenoid fossa, but the socket is rather shallow, more like a saucer than an actual socket, which allows for more freedom of movement, but also relies almost entirely on ligaments and muscles to keep the joint together. In some circumstances, these ligaments and muscles stretch too much, allowing the ball to slip out of the socket, which does not itself firmly hold the ball in place. When the stretching forces are removed from the joint, the ligaments snap back to their original length, pulling the ball back toward the shoulder, but, often, not into the socket, resulting in a dislocated shoulder joint. The ball cannot slip back into the socket on its own because the ball portion of the joint has been pulled up into a space in the shoulder that is more inward of the shoulder than the socket portion of the joint. The only way to relocate the ball into the shoulder joint is to provide a smooth constant traction or stretching force onto the arm pulling the arm away from the shoulder. A force of perhaps fifty pounds, more or less, is typically required to stretch the ligaments and muscles and to pull the ball away from the shoulder joint enough so that the ball reseats in the socket when the traction force is gradually reduced. It is not necessary to manipulate the arm to any significant extent to reduce the dislocated shoulder because the natural place for the ball is in the socket and ligaments are naturally designed to hold the joint together.

Dislocated shoulders are fairly common. Although often associated with sports players, this injury is common among the general population. It is caused by some trauma that stretches the ligaments too much. Frequently, for example, it seems that if a person falls over backward and extends his arms downward ly behind his back to break his fall, a dislocated shoulder frequently results, but can also result from as simple an act as shrugging to put on a coat. Dislocated shoulders are also fairly common in adults who have suffered a previous dislocated shoulder, since the stretched ligaments may not return to their original length and may not be as strong as they should be.

Since a dislocated shoulder is very painful and obviously the shoulder joint cannot operate properly when the ball and socket are not mated, it is essential that the joint be restored to its natural condition, that is, reduced, as quickly as possible with as little pain as possible. A number of prior art techniques and devices to accomplish shoulder reduction have been developed and a number have led to issued patents, such as those discussed below.

U.S. Pat. No. 5,788,659, issued to Haas on Aug. 4, 1998, discloses a “Shoulder Traction Device for Relocating a Dislocated Shoulder” comprising a strap that is passed under the armpit of the affected arm and is held by an assistant, who has a portion of the strap wrapped around his waist and a separate arm isolation component for isolating the elbow from the injured shoulder joint. The attending physician pulls on and manipulates the dislocated shoulder. The patent covers the specific structure of the various straps and adjustment buckles, and so forth. This invention requires two people to effect the reduction.

U.S. Pat. No. 3,680,552, issued to Bell et al. on Aug. 1, 1972, discloses a “Traction Splint” comprising a traction splint for the arm or the leg having a pallet or cradle and a cuff that attaches an upper portion of the limb to the cradle and a second cuff attached to the lower end of the limb. The second cuff is attached to a cable that is manually pulled to apply traction to the limb. A latch allows the cable to be held under tension and allows that tension to be quickly and easily released. The device is intended to relieve the pain of a broken bone by applying some traction. This device is designed specifically to provide some tension on a broken bone to relieve pain prior to setting the bone.

U.S. Pat. No. 3,477,428, issued to Hare on Nov. 11, 1969, discloses a “Combined Splint and Traction Device” comprising a splint cradle that is strapped to a leg and has a roughly semi-circular proximal end that fits against the patient's hip. A strap is wrapped around the ankle and foot and is connected to a rachet mechanism that pulls on the ankle strap to apply traction to the leg. The rachet mechanism is connected to the frame of the distal end of the splint. The tension on the ankle strap can be easily and quickly released by releasing the rachet pawl. This device is designed specifically for a hip joint and does not address reduction of the shoulder joint and is designed to provide traction to a broken leg bone.

U.S. Pat. No. 2,590,739, issued to Wagner et al. on Mar. 25, 1952, discloses an “Orthopedic Bone Aligning and Fixing Mechanism” a mechanical device with a large frame cantilevered from the patient's bed and having many joints and adjustments. The device is designed to allow end bones, such as the humerus, to be set after being broken. The device further comprises a strap wrapped around the patient's chest adjacent to the armpit on the affected side and secured to a rigid upstanding post 60 adjacent to the opposite side of the patient's body. This device requires the patient to be placed on a bed and requires a substantial amount of dedicated space for its complex and large apparatus.

U.S. Pat. No. 2,515,590, issued to Chaffin on Jul. 18, 1950, discloses an “Apparatus for Tensioning an Arm” designed to assist in setting fractures in the forearm or certain other injuries in the forearm. The device includes a strap about the biceps portion of the arm and fastened to a support or held by a person. A sleeve is connected to the wrist and is connected to a cable and pulley system. An anchoring cable is fastened to a fixed support. This device allows for applying tension along a line from the back to the front of the patient, which is not the direction of tension required for shoulder reduction and its use requires two medical personnel.

In many medical facilities, particularly in rural areas, on ambulances, and so forth, there may not be two skilled medical workers to attend to a single patient; there may not be enough space to dedicate a significant amount of room to a specialized shoulder reduction area; there may not be enough financial resources for an expensive complex shoulder reduction system. Further, considerable physical strength and stamina are needed to reduce the dislocated shoulder using prior art techniques and the available workers may well not have the strength needed.

Therefore, a need exists for a shoulder reduction and splint device that can be operated by a single skilled medical worker; that does not require any significant amount of space, during either use or storage; that is inexpensive to purchase and maintain; and that does not require significant physical strength to use successfully.

### BRIEF SUMMARY OF THE INVENTION

Accordingly, it is a primary object of the present invention to provide a shoulder reduction device and splint device that can be successfully and easily operated by a single skilled medical worker without assistance and without any secondary anchoring system or strap.

It is another object of the present invention to provide a shoulder reduction and splint device that does not require any significant amount of space during either use or storage.

It is another object of the present invention to provide a shoulder reduction and splint device that is inexpensive to purchase and to maintain.

It is another object of the present invention to provide a shoulder reduction and splint device that does not require significant physical strength to use successfully.

These and other objects of the present invention are achieved by providing a staff having a distal end that is placed in the armpit under the patient's dislocated shoulder joint and a proximal end that is braced against the stomach or torso of a skilled medical worker. The distal end is preferably provided with a cross member or horizontal portion that is padded or cushioned to reduce the pressure in the patient's armpit. A wrist cuff is attached to the wrist of the affected arm and adjusted so that the patient's hand cannot pass through it. A traction cord is fastened to the wrist cuff and includes a pulling end having a handle, which the skilled medical worker pulls on to apply the traction needed to reduce the dislocated shoulder. The wrist cuff and traction cord may be separate from the staff.

Preferably, however, the traction cord includes a fixed end that is attached to the staff adjacent to the proximal end of the staff and is operatively routed through a rachet mechanism so that the skilled medical worker can relax during the reduction without losing the traction applied prior to relaxing. The traction is then maintained by the portion of the traction cord between the fixed end of the traction cord and the rachet mechanism.

The length of the staff can be adjusted to facilitate its use with arms of greatly differing lengths and to suit the needs of different medical workers. The length of the staff can be adjusted by telescoping staff sections that can be fixed into specific lengths by a spring-loaded protruding button that projects through a selected length adjustment aperture selected from a row of spaced adjustment apertures. Alternatively or in addition, the length of the staff can be adjusted by turning a long screw that runs the length of a lower section of the staff and is received by a threaded nut fixed in a distal end of a middle section of the staff.

Other objects and advantages of the present invention will become apparent from the following description taken in connection with the accompanying drawings, wherein is set forth by way of illustration and example, the preferred embodiment of the present invention and the best mode currently known to the inventor for carrying out his invention.

### BRIEF DESCRIPTION OF THE SEVERAL VIEWS OF THE DRAWINGS

FIG. 1 is a perspective view of a prior art shoulder reduction in progress in which the patient is supine.

FIG. 2 is a top plan view, that is, looking down on the supine patient, of a prior art shoulder reduction in progress in which the patient is supine.

FIG. 3 is a perspective view of the preferred embodiment of a shoulder reduction device in accordance with the present invention.

FIG. 4 is an enlarged fragmentary perspective view of the proximal (lower) end of the staff portion of the shoulder reduction device of FIG. 3 showing the attachment of the traction cord to the staff.

FIG. 5 is a perspective view of a shoulder reduction in progress utilizing the preferred embodiment of the present invention with the patient in a supine position.

FIG. 6 is a perspective view of an alternative embodiment of a shoulder reduction device in accordance with the present invention.

FIG. 7 is an enlarged fragmentary view of the lower portion of the staff of FIG. 6 showing the screw mechanism for adjusting the length of the staff.

FIG. 8 is a perspective view of an alternative embodiment of the shoulder reduction device of FIG. 3 in which the traction cord and associated parts are not connected to the staff.

### DETAILED DESCRIPTION OF THE INVENTION

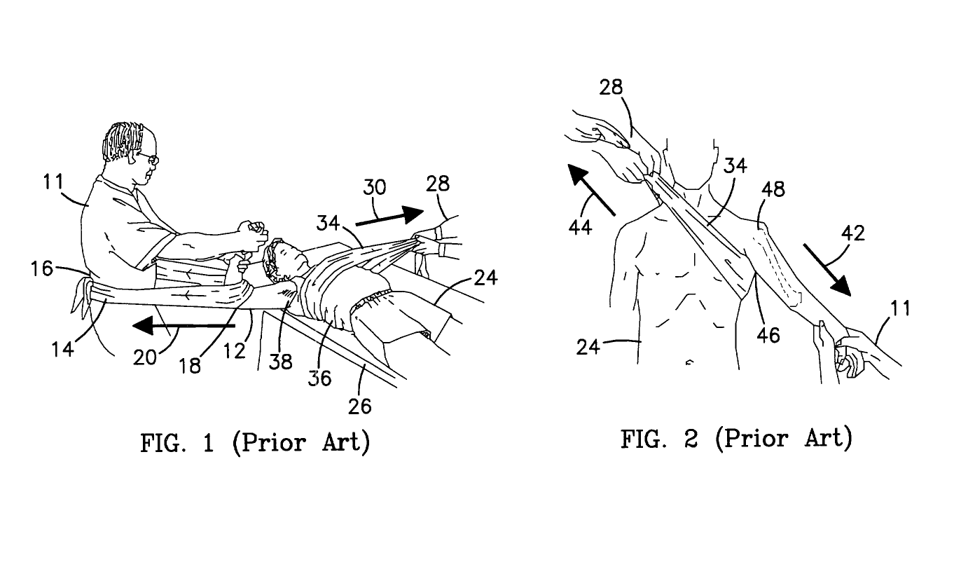

Referring to FIGS. 1, 2 a standard prior art sling technique is illustrated. As shown in FIG. 1, a skilled medical worker 11, such as a physician or physician's assistant, holds the patient's affected (right) arm 12 bent upwardly at the elbow, while a clothe sling 14 is wrapped around the medical worker's waist 16 and the patient's arm 12 at the elbow 18. The medical worker 10 applies a force in the direction of the arrow 20 basically by leaning backward. In order that the for force in the direction of the arrow 20 to apply traction to the shoulder and not simply pull the patient 24 off the bed 26, an assistant 28, who need not be skilled, pulls in more or less the opposite direction in the direction of the arrow 30 on the assistant's sling 34, which is wrapped about the patient 24 at his torso 36 under his armpit 38. As shown in FIG. 1, the patient 24 is supine.

Referring to FIG. 2, a similar procedure with the patient 24 in a supine position is illustrated, but the skilled medical worker 11 does not use a sling and simply pulls downwardly along the natural line of the patients affected (left) arm 40, that is, along the arrow 42, while the assistant 28 pulls in basically the opposite direction along the arrow by applying force to the assistant's sling 34, which is looped under the patient's left armpit 46.

In the case of either FIG. 1 or 2, two workers are required. Further it is evident from the drawings that the forces applied by the skilled medical worker 11 and the assistant 28 are not operating in opposite directions along the same line. Rather, the force applied by the assistant 28 are along a line that is downwardly offset from the force applied by skilled medical worker 11, thereby resulting in shear forces in the affected shoulder joint 48 of the patient 24. Ideally, the opposing forces should be as nearly along the same line as possible to minimize the resulting pain, asymmetrical stretching of ligaments on different sides of the affected shoulder joint 48 and to minimize the stretching required for the shoulder reduction. The prior art technique of both FIGS. 1, 2 may be used only when the patient 24 is supine. In virtually every case the patient will be supine and this is also the case with the shoulder reduction device 10 disclosed herein, but it would be possible to use the device 10 when the patient is standing.

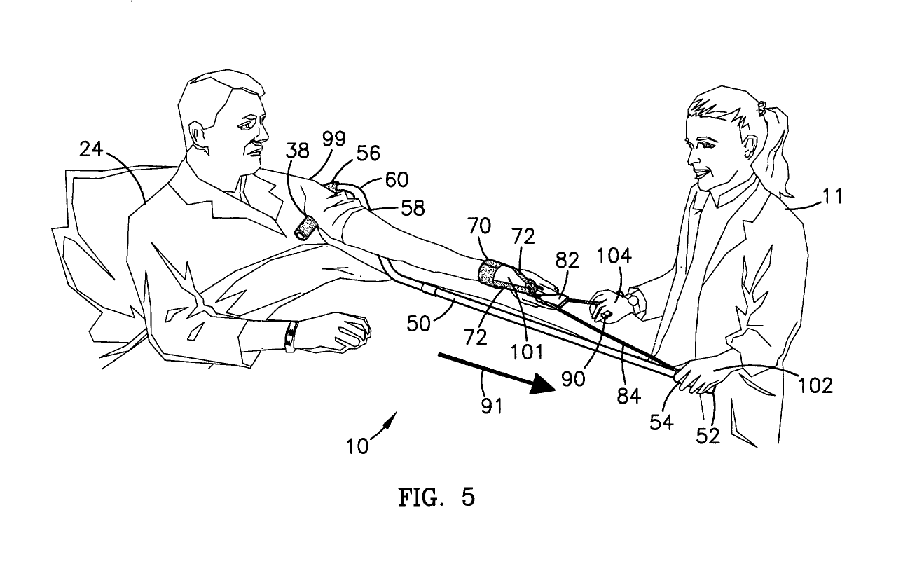

Referring now to FIG. 3, there is shown the preferred embodiment of a shoulder reduction device 10 according to the present invention, which includes an elongated tubular staff 50 having a proximal end 52 (closest to the skilled medical worker 11 during use) covered with a padded sleeve 54 such as a foam grip, to reduce the pressure when the proximal end 52 is pushed into the skilled medical worker's stomach during use and to provide a high-friction grip. A distal end 56 (farthest from the skilled medical worker 11 during use) includes a crooked portion 58 bent outwardly from the general line of the elongated tubular staff 50, preferably aluminum, and an inward bend 60 leading to a perpendicular portion, or armpit brace, or force distribution member or portion, 62, which is substantially straight, but which includes a dip 64, which distributes the force applied by the skilled medical worker 11 over a large area of the patient's armpit 38, 46 to reduce the pressure relative to a staff 50 alone and thereby reduce any discomfort to the patient 24. The perpendicular portion 62 is perpendicular to the general line or longitudinal axis of the elongated tubular staff 50, fits comfortably into the armpit of the patient 24 and is covered by a tubular cushioned sleeve 66 to improve patient comfort and to increase the holding friction between the shoulder reduction device 10 and the patient's armpit 38, 46 under the affected shoulder. Bending the elongated tubular staff 50 so that it also includes the armpit brace 62 allows a single piece of tubular material to serve as the staff and an armpit brace, simplifying construction. Alternatively, the armpit brace could be a separate member, such as a cross member, crutch top or the like fastened to a straight staff, but this would generally require two separate pieces that would be assembled (unless the entire assembly were molded). Attached to the elongated tubular staff 50 approximately adjacent to the crooked portion 58 is an arm cuff 68, which is optionally used to hold the patient's upper arm against the elongated tubular staff 50 to insure that the line of the patient's arm lies along the line of the elongated tubular staff 50 (See FIG. 5). The arm cuff 68 is fastened to itself after being passed around the patient's arm by mating hook and loop fasteners, buckles or the like.

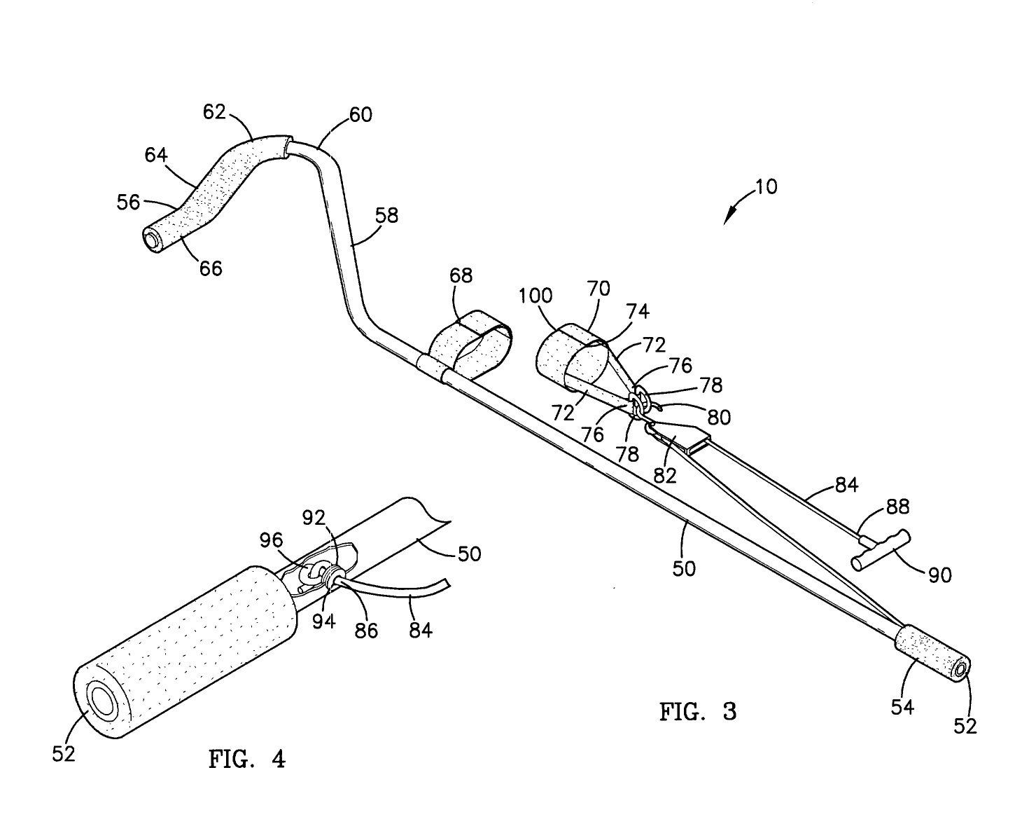

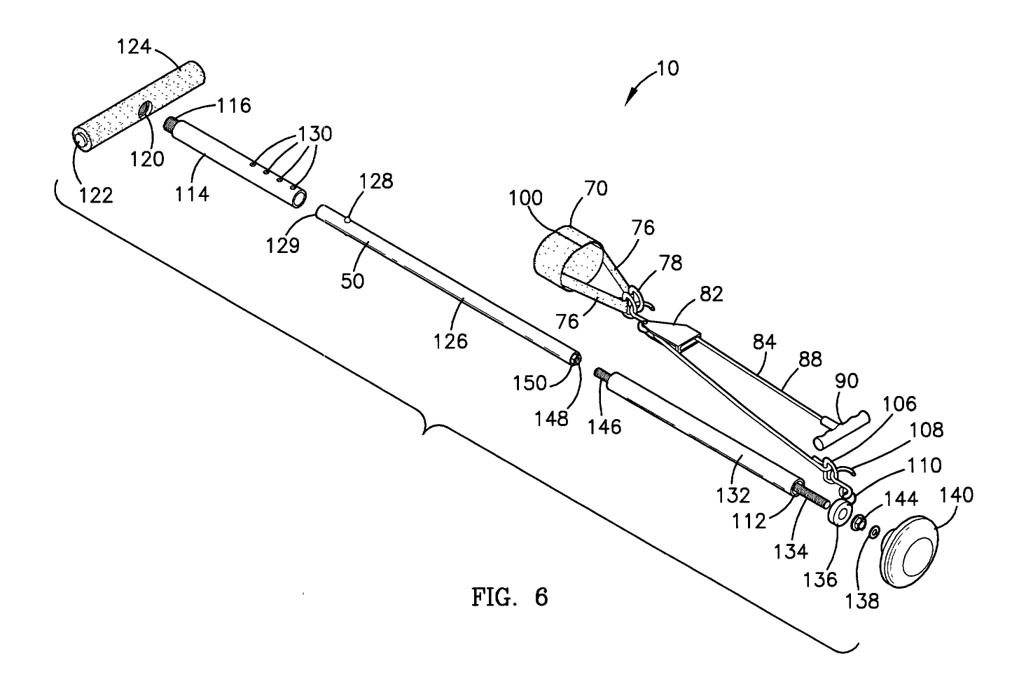

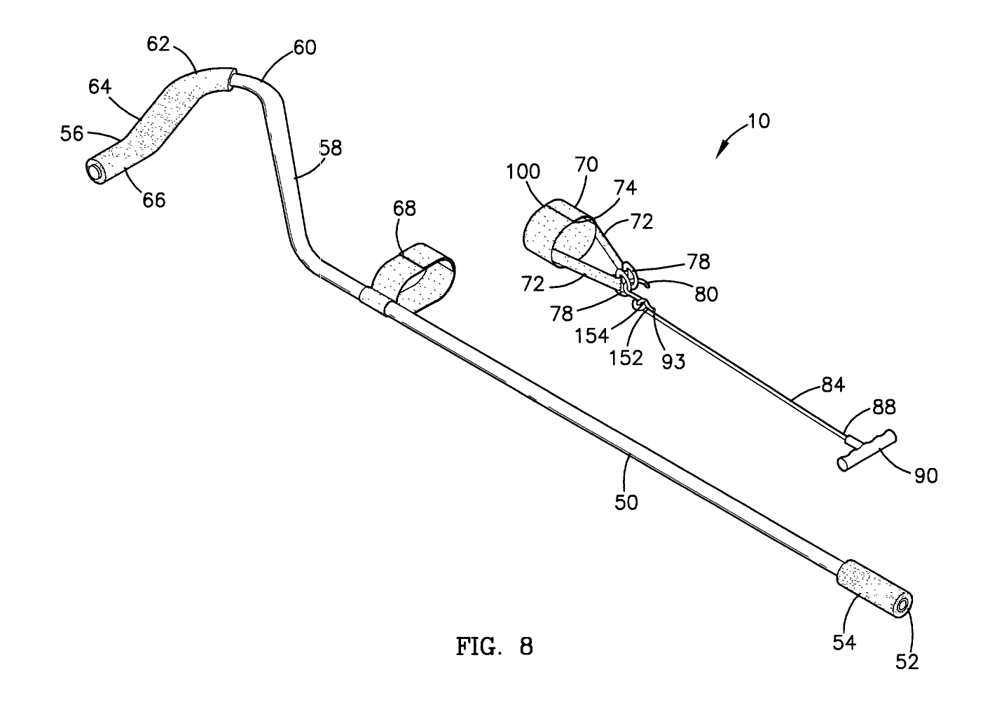

Still referring to FIG. 3, a substantially cylindrical wrist cuff 70 is connected to a pair of force transmitting straps 72, which are fastened to a lower edge 74 of the wrist cuff 70 by sewing or the like and which are located across a diameter of the wrist cuff 70 from each other. A proximal end 76 of each of the two force transmitting straps 72 is folded over itself and a D-ring 78 and the two resulting layers of straps are sewn together or otherwise fastened to secure the D-rings 78 to the straps 72. An S-hook 80, or other type hook or fastener, attached to a quick-release rachet mechanism 82, such as that described in U.S. Pat. No. 5,368,281, issued Nov. 29, 1994 to Skyba, which is hereby incorporated by reference, is inserted into the D-rings 78. Alternatively, a simple pulley without a rachet mechanism may be used, but this option requires a stronger and steadier hand to apply and maintain the proper amount of traction. A traction cord or rope 84 is operatively routed or threaded through the rachet mechanism and includes a fixed end 86 that is seated in and fixed to the proximal end 52 of the elongated tubular staff 50 and a pulling end 88, which terminates in a T-handle 90 that is pulled by the skilled medical worker 11. Using the rachet mechanism 82 allows the skilled medical worker 11 to relax after applying a certain amount of traction to the traction cord 84 (and hence to the affected shoulder) without having the traction forces released. As shown in FIG. 4, the fixed end 86 of the traction cord 84 is threaded through a grommet 92, seated in an aperture 94 adjacent to the distal end 56 of the elongated tubular staff 50 and is tied in a knot 96 to retain it in the staff 50. The T-handle 90 is similarly fastened to the other end of the traction cord 84. Alternatively, the traction cord may be simply fastened to the wrist cuff 70 as described above without being connected to the staff 50 at all or to any pulley or rachet mechanism, as shown in FIG. 8, in which case, the skilled medical worker 11 simply pulls on the traction cord 84 at the T-handle 90. In this case, the traction cord 84 and wrist cuff 70 are related to the staff 50 but are not a part of it and are not connected to it. In the embodiment of FIG. 8, either the staff 50 of FIG. 3 or the length adjustable staff 50 of FIG. 6 may be utilized. The length of the staff 50 as shown in FIG. 3 is fixed, although it may be made adjustable as shown in FIG. 6.

Referring now to FIG. 5, in use, the skilled medical worker 11 places the proximal end 52 of the staff 50 against her stomach 98 and the distal end 56 in the patient's armpit under the affected joint (left shoulder 99), applies the wrist cuff 70 to the patient' wrist using the hook and loop fasteners 100, and then pulls on the T-handle 90, while bracing the staff 50. The wrist cuff 70 is closed to a diameter that is too small to allow the patient's hand 101 to pass through it, allowing traction to be pulled on the affected joint. Typically, her 11 right hand 104 holds and pulls the T-handle 90, while her left hand 102 grips the padded sleeve 54 to stabilize the staff 50. Traction or traction force is defined as a force pulling on the patient's arm away from the patient's body, more or less along the line of the arrow 91 in FIG. 5 and may or may not lie along the line of actual force applied by the skilled medical worker 11, since that force may be translated along a different direction by the traction cord 84 and associated hardware.

Referring now to FIG. 6, an alternative embodiment of the shoulder reduction device 10 features a staff 50 whose length can be adjusted to accommodate different lengths of arms of patients or doctors by utilizing a telescoping staff 50 consisting of an upper staff member section 114, a middle staff member section 126 and a lower staff member section 132. This embodiment is similar to the preferred embodiment of FIG. 3, but also includes a length-adjustable staff 50. The wrist cuff 70, attached force transmitting straps 72, D-rings 78, rachet mechanism and traction cord 84 are the same and operate the same as in the embodiment shown in FIG. 3. The fixed end 86 of the traction cord 84 is attached to a D-ring 106, which is slipped over an end of an S-hook 108, which is seated in a aperture 110 in the staff 50 adjacent to the proximal end 112. The shaft 50, which may be a tubular shaft having a cylindrical cross section as shown, a square cross section or any other desired cross section, is provided in three sections, allowing for gross and fine adjustment of the length of the shaft 50 as used.

Still referring to FIG. 6, an upper staff section 114 includes a distal end 116 that is a projecting threaded stud 118, adapted to be received by a threaded aperture 120 in the middle of a force distribution member 122, which is covered and padded by a cushion member 124, thereby forming a T-shaped structure. The force distribution member 122 is seated in the affected armpit 99 (see FIG. 5) during reduction.

Still referring to FIG. 6, a middle staff section 126 includes a conventional spring-loaded protruding locking member 126 that can be depressed to be level with the outer surface of the middle staff section 126, while the upper staff section 114 is slid along and concentric with the middle staff section 126 until the desired length adjustment aperture 130 is located over the protruding locking member 126, which then springs up, locking the upper staff section 114 and the middle staff section 126 together at a desired length. The protruding locking member 126 is fixed to the middle staff section 126 adjacent to a distal end 129 of the middle staff section 126. Four spaced length adjustment apertures 130 are provided, all aligned along a straight line. This provides for a gross adjustment of the length of the staff 50.

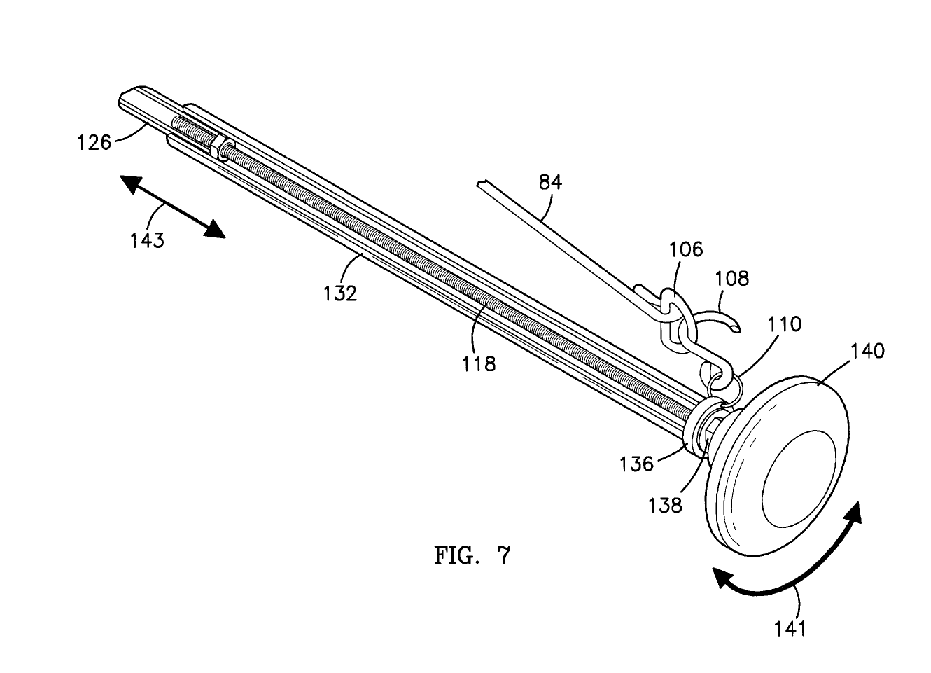

Referring to FIGS. 6, 7, fine length control of the staff 50 is accomplished by a screw mechanism utilizing the threaded stud 134. A lower staff member section 132 includes a proximal end 112 having a protruding threaded stud 134 that accepts a cylindrical drilled spacing collar 136, washer 138 and a padded knob 140, which includes a threaded bore that is screwed onto the threaded stud 134. The threaded stud 134 extends throughout the length of the lower staff member section 132 and projects outwardly from both ends of the lower staff member section 132. The spacing collar 36 is restrained in the distance it can move along the threaded stud 134 by the stop member 144 fixed to the threaded stud 134. The threaded stud 134 extends though the entire length of the lower staff member section 132 and includes a distal end portion 146 that is received by a threaded nut 148 fixed into a proximal end 150 of the middle staff section 126. Rotating the padded knob 140 in one direction (typically clockwise as seen from the viewpoint of the skilled medical worker 11 in FIG. 5) thereby draws the threaded stud 134 farther into the middle staff section 126, thereby shortening the length of the complete staff, while rotating the padded knob 140 in the opposite direction shortens the overall length of the staff 50, thereby providing fine adjustment of the length of the staff 50. Rotation of the padded knob in either direction is indicated by the arrow 141, which is translated by the screw 118 into the linear movement of the middle staff member 126 section 126 as shown by the arrow 143 and drawing the middle staff member 126 into the lower staff member section 132 or pushing it away from the lower staff member section 132, thereby shortening or lengthening the overall elongated tubular staff 50, respectively.

Referring to FIG. 8, an alternative embodiment of the shoulder reduction device 10 is illustrated in which the traction cord 84 is connected to a T-handle 90 at the pulling end 88 of the traction cord 84 and the distal end 93 is folded over itself and sewn or the like along the seam 152 to form the loop 154, which is slipped over the S-hook 80, and which is attached to the wrist cuff 70 in the same fashion as described above in connection with FIG. 3. In this embodiment, the skilled medical worker 11 attaches the wrist cuff 70 to the patient's wrist on the hand of the affected arm, braces the staff 50 between her body and the patient's armpit, and pulls on the T-handle 90, as described above. This embodiment is simpler to make but is somewhat harder to use because the staff 50 does not assist in properly aligning the traction forces and does not allow the skilled medical worker 11 to relax the arm that is applying traction to the arm. The ability for the skilled medical worker to relax during the procedure is a principal benefit of utilizing the rachet mechanism 82 in the embodiment illustrated in FIGS. 3, 5, and 6.

In using either embodiment of the embodiment of the shoulder reduction device 10, it is not necessary for the skilled medical worker 11 to hold or touch the affected arm or shoulder because the shoulder joint does not need guidance in order to accomplish reduction—it only needs firm sufficient stretching of the joint ligaments, which will snap the joint back together once the ligaments have been stretched sufficiently. Since it is highly desirable to release the traction on the joint quickly after reduction is achieved, a quick release rachet mechanism 82 is preferred in the preferred embodiment. Either embodiment of the shoulder reduction device 10 may be used for reduction of either the left-hand or right-hand shoulder joint. Neither embodiment of the shoulder reduction device 10 has a handedness, so the skilled medical worker 11 can use either hand to pull on the T-handle 90 and either hand to hold the shoulder reduction device 10, as desired. Further, only one person, a skilled medical worker, is needed to operate the shoulder reduction device 10 successfully.

It has been found that using the shoulder traction device 10 can dramatically reduce the time needed for reduction of a dislocated shoulder because the rachet mechanism 82 allows the skilled medical worker 11 to apply steady even force to the affected shoulder joint. When the two-person prior art technique is used, one person may relax a bit or may pull a little harder and its very difficult, if not impossible, for the other worker to compensate for the changing forces, resulting in the uneven application of force to the dislocated shoulder. In one actual case, a physician and an assistant unsuccessfully struggled for more than two hours to reduce a dislocated shoulder and then a single skilled medical worker utilizing the shoulder reduction device according to the present invention was able to reduce the dislocation in about two and one-half minutes. The key to a quick, minimally painful shoulder joint reduction is the application of steady force that can be incrementally increased and well-controlled until the force required to draw the humeral head to relocate back into its socket, which is greatly facilitated by the shoulder reduction device 10. The shoulder reduction device 10 can also be used to stabilize a fractured humerus by using the device 10 as a splint with straps wrapped around the fractured limb and applying traction to hold the fractured bone ends apart to alleviate pain and further damage to the bones until the fracture can be set.

While the present invention has been described in accordance with the preferred embodiments thereof, the description is for illustration only and should not be construed as limiting the scope of the invention. Various changes and modifications may be made by those skilled in the art without departing from the spirit and scope of the invention as defined by the following claims.

I claim:

1. A shoulder reduction device comprising:
a. a staff having a proximal end and a distal end; and
b. related means for applying traction to a shoulder of a patient.

2. A shoulder reduction device in accordance with claim 1 further comprising means for attaching said traction applying means to said staff adjacent to said distal end of said staff.

3. A shoulder reduction device in accordance with claim 1 further comprising a force distribution member adjacent to said distal end of said staff.

4. A shoulder reduction device in accordance with claim 3 wherein said force distribution portion further comprises a T-shaped distal end of said staff.

5. A shoulder reduction device in accordance with claim 3 wherein said force distribution portion further comprises an outwardly bent crooked portion of said staff adjacent to said distal end of said stall and an inward bend providing an armpit brace perpendicular to a longitudinal axis of said staff.

6. A shoulder reduction device in accordance with claim 1 further comprising means for lengthening or shortening said staff.

7. A shoulder reduction device in accordance with claim 6 wherein said lengthening or shortening means further comprises a telescoping staff.

8. A shoulder reduction device in accordance with claim 6 wherein said lengthening or shortening means further comprises a plurality of length adjustment apertures in an upper staff section and a protruding locking mechanism fixed to a middle staff section of said staff adjacent to a distal end of said middle staff section.

9. A shoulder reduction device in accordance with claim 6 wherein said lengthening or shortening means further comprises a threaded stud extending through a lower staff member section and having a distal end portion received by a threaded nut fixed into a proximal end of said middle staff section.

10. A shoulder reduction device in accordance with claim 1 further comprising means for transmitting a traction pulling force toward the skilled medical worker to a wrist strap secured to the wrist of the patient and to a point adjacent to said distal end of said staff.

11. A shoulder reduction device in accordance with claim 10 wherein said traction applying means further comprises a wrist cuff adapted to be secured to the wrist of a patient, said wrist cuff being attached to a traction cord, said traction cord having a fixed end fixed to said staff adjacent to a proximal end of said staff and a pulling end manipulated by a skilled medical worker.

12. A shoulder reduction device in accordance with claim 11 further comprising a rachet mechanism connected to said wrist cuff and means for receiving said traction cord in said rachet mechanism.

13. A shoulder reduction device in accordance with claim 11 further comprising a handle connected to said pulling end of said traction cord.

14. A shoulder reduction device comprising:
a. a staff having a proximal end and a distal end; and
b. means for applying traction to a shoulder of a patient, said traction applying means fixed to said staff adjacent to said distal end of said staff.

15. A shoulder reduction device in accordance with claim 14 further comprising a force distribution member adjacent to said distal end of said staff.

16. A shoulder reduction device in accordance with claim 15 wherein said force distribution portion further comprises an outwardly bent crooked portion of said staff adjacent to said distal end of said stall and an inward bend providing an armpit brace perpendicular to a longitudinal axis of said staff.

17. A shoulder reduction device in accordance with claim 15 further comprising means for lengthening or shortening said staff.

18. A shoulder reduction device in accordance with claim 15 wherein said traction applying means further comprises a wrist cuff adapted to be secured to the wrist of a patient, said wrist cuff being attached to rachet mechanism having a traction cord operatively routed through it, said traction cord having a fixed end fixed to said staff adjacent to a proximal end of said staff and a pulling end manipulated by a skilled medical worker.

19. A shoulder reduction device comprising:
a. a staff having a proximal end and a distal end;
b. means for distributing forces in the patient's armpit, said force distribution means connected to said staff adjacent to said distal end of said staff; and
c. means for applying traction to a shoulder of a patient, said traction applying means fixed to said staff adjacent to said distal end of said staff.

20. A shoulder reduction device in accordance with claim 19 further comprising wherein said traction applying means further comprises a wrist cuff adapted to be secured to the wrist of a patient, said wrist cuff being attached to rachet mechanism having a traction cord operatively routed through it, said traction cord having a fixed end fixed to said staff adjacent to a proximal end of said staff and a pulling end manipulated by a skilled medical worker.
